(19) United States
(12) Patent Application Publication
(10) Pub. No.: US 2021/0035678 A1
(43) Pub. Date: Feb. 4, 2021

(54) VISUAL REPRESENTATION OF IMAGE ANALYSIS OUTPUT IN AN ALTERNATIVE VISUALIZATION OF DIGITAL PATHOLOGY IMAGES

(71) Applicant: KONINKLIJKE PHILIPS N.V., Eindhoven (NL)

(72) Inventors: ALBERTO CORVO, EINDHOVEN (NL); MARC VAN DRIEL, NIJMEGEN (NL); MICHEL WESTENBERG, EINDHOVEN (NL)

(21) Appl. No.: 16/942,829

(22) Filed: Jul. 30, 2020

Related U.S. Application Data
(60) Provisional application No. 62/880,807, filed on Jul. 31, 2019.

(51) Int. Cl. G16H 30/40; G06F 3/0481; G06T 11/00; G06F 3/0484; G16H 70/60; G16H 40/67

(52) U.S. Cl. CPC G16H 30/40; G06F 3/0481; G06T 11/001; G06T 2200/24; G16H 70/60; G16H 40/67; G06F 3/04842

(57) ABSTRACT

In one embodiment, a method performed by one or more processors, the method comprising: receiving information about tissue or cell areas of a single digital pathology image; and visually representing each of the tissues or cell areas as a proportion of all of the tissue or cell areas using one or more respective, nested, interactive areas located entirely within a single rectangle, the nested areas proportional to the respective proportions of the tissue areas.

### FIELD OF THE INVENTION

The present invention is generally related to digital pathology, and more particularly, digital pathology imaging.

### BACKGROUND OF THE INVENTION

Digital pathology images are used as input for image analysis algorithms that produce a vast amount of information. Tissue regions are typically detected and measured. Tissue-specific detectors (e.g., using automated pattern recognition) are improving in the recognition of tissue variations and/or other tissue characteristics, and provide accurate tissue regions. Once tissue regions are detected, these are labelled according to one of a plurality of tissue types (e.g., tumor, stroma, fat, and other histological components, which are usually employed by pathologists in a diagnostic practice). Such information may be valuable to pathologists and researchers in many ways, particularly in visualizations given the display of digital pathology images content on a Graphical User Interfaces (GUI).

Digital pathology images are often collected in vast amounts, and are usually represented on a GUI by means of thumbnails, which do not provide much insight, particularly if users are not expert pathologists. A large quantity of thumbnails can easily become confusing, and require large displays to generate an image gallery that allows users to recognize image content. Digital pathology images generally provide a user with biological/tissue insights only once the whole-slide image format is accessed (and not at the thumbnail level).

### SUMMARY OF THE INVENTION

In one embodiment, a method performed by one or more processors, the method comprising: receiving information about tissue or cell areas of a single digital pathology image; and visually representing each of the tissue or cell areas as a proportion of all of the tissue or cell areas using one or more respective nested, interactive areas located entirely within a single area, the nested areas proportional to the respective proportions of the tissue or cell areas.

These and other aspects of the invention will be apparent from and elucidated with reference to the embodiment(s) described hereinafter.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the present invention. Moreover, in the drawings, like reference numerals designate corresponding parts throughout the several views.

FIG. 1 is a schematic diagram that illustrates an example environment in which an example pathology imaging visualization system is used, in accordance with an embodiment of the invention.

FIG. 2 is a block diagram that illustrates an example computing device used to implement one or more functionality of a pathology imaging visualization system, in accordance with an embodiment of the invention.

FIG. 3 is a schematic diagram that conceptually illustrates an example pathology imaging visualization method, in accordance with an embodiment of the invention.

FIG. 4 is a schematic diagram that illustrates an example visualization for plural digital pathology images, in accordance with an embodiment of the invention.

FIG. 5 is a schematic diagram that conceptually illustrates an example visualization of tissue types for a digital pathology image for an example pathology imaging visualization system, in accordance with an embodiment of the invention.

FIGS. 6A-6B are schematic diagrams that illustrate example visualizations based on different staining colors for an example pathology imaging visualization system, in accordance with an embodiment of the invention.

FIG. 7 is a schematic diagram that conceptually illustrates interactive functionality based on a type of user input enabled by an example pathology imaging visualization system, in accordance with an embodiment of the invention.

FIGS. 8A-8B are schematic diagrams that illustrate example symbols used to visually represent various tissue content information for an example pathology imaging visualization system, in accordance with an embodiment of the invention.

FIGS. 9A-9E are schematic diagrams that illustrate different area representations for example visualizations that may be used for an example pathology imaging visualization system, in accordance with an embodiment of the invention.

FIG. 10 is a flow diagram that illustrates an example pathology imaging visualization method, in accordance with an embodiment of the invention.

### DETAILED DESCRIPTION OF EMBODIMENTS

Disclosed herein are certain embodiments of a pathology imaging visualization system and method (collectively hereinafter referred to as a pathology imaging visualization system) that provide a specific type of visualization that may be used to provide immediate insights and awareness of tissue and/or cellular content in digital pathology images acquired from digital pathology slides. In one embodiment, a pathology imaging system is configured to display automatically detected tissue and/or cellular area information using nested, interactive (e.g., user selectable) areas (e.g., nested rectangles, or other shapes in some embodiments), and in particular, provide a tree-map like visualization that encodes percentages of automatically detected tissue and/or cell types with nested rectangles inside a rectangular shape that represents the digital pathology image (e.g., whole slide image). In some embodiments, the visualization includes a color component, where the colors are intended to aid users to recognize specific biological content by resembling the colors of such features. In some embodiments, other tissue and/or cellular features or characteristics may be visually represented, as explained below.

Digressing briefly, thumbnails used in digital pathology image analysis are sparse in insightful information, and necessitate the user interacting with each thumbnail (e.g., selection) to expand the thumbnail image into the full-blown image, resulting in more time expenditure, complexity in visualization (e.g., more screen renderings), and/or additional compute resources (e.g., additional processing cycles). It would be helpful to be able to retain the size of the thumbnail images for quick assessment of tissue content and/or tissue content distribution yet enable the user to ascertain more information and/or insights to facilitate the task of pathologists and other users in understanding the digital image content (e.g., tissue content, cell content). In certain embodiments of a pathology image visualization system, the vast amount of images collected through digital pathology imaging are conveniently represented by plural thumbnail-sized images, yet with more information to facilitate the analysis by a user of tissue content.

The images presented by an embodiment of a pathology imaging visualization system are configured in a tree-map like manner. Tree-maps comprise 2D space-filling visualizations that are commonly used in the information visualization domain to depict large amounts of hierarchical data. In certain embodiments of a pathology imaging visualization system, the tree-maps represent hierarchical datasets, within which nodes are displayed by subdividing a rectangular area in smaller, nested rectangular areas proportional to the value of the node. These tree maps can be convenient to make efficient use of display space on a GUI. Also, a tree-map can enable exploration of the dataset hierarchy by means of interaction on each node (e.g., via a mouse click). Tree-maps are intuitive to common users as they exploit the perceptive abilities of human brain in recognizing area. Colors may also be used to highlights relationships between nodes and hierarchy edges. Accordingly, the visualizations provided through certain embodiments of a pathology imaging visualization system improve GUI technology in the pathology field by providing immediate insights and awareness of tissue and/or cellular content in digital pathology images acquired from digital pathology slides in a meaningful, informative yet quicker way, while reducing the complexity associated with conventional GUI systems and hence providing ease of use to the user.

Having summarized certain features of a pathology imaging visualization system of the present disclosure, reference will now be made in detail to the description of a pathology imaging visualization system as illustrated in the drawings. While a pathology imaging visualization system will be described in connection with these drawings, there is no intent to limit it to the embodiment or embodiments disclosed herein. For instance, whilst many of the examples described in this disclosure focus on diagnostic aspects, it should be appreciated by one having ordinary skill in the art that some embodiments of a pathology imaging visualization system have more general applicability, such as for providing tissue and/or cell content information (e.g., used to characterize tissues and interactions between cells and tissue regions), or in general, providing microenvironment information from tissue and/or cell analysis in clinical, research environments, and/or generally, digital pathology and/or tissue and cell analysis. Also, though the example illustrations of visualizations depicted in the attached drawings focus on the use of rectangles nested within a single rectangle, it should be appreciated by one having ordinary skill in the art, in the context of the present disclosure, that other types of (geometric) areas (e.g., other polygonal shapes, including circles, etc.), including other mechanisms for visually representing tissue and/or cell characteristics (e.g., using differences in size, colors, patterns, associated symbols, etc.) used in conjunction with these areas, may be used in some embodiments. Further, although the description identifies or describes specifics of one or more embodiments, such specifics are not necessarily part of every embodiment, nor are all of any various stated advantages necessarily associated with a single embodiment. The intent is to cover all alternatives, modifications and equivalents included within the principles and scope of the disclosure as defined by the appended claims. As another example, two or more embodiments may be interchanged or combined in any combination. Further, it should be appreciated in the context of the present disclosure that the claims are not necessarily limited to the particular embodiments set out in the description.

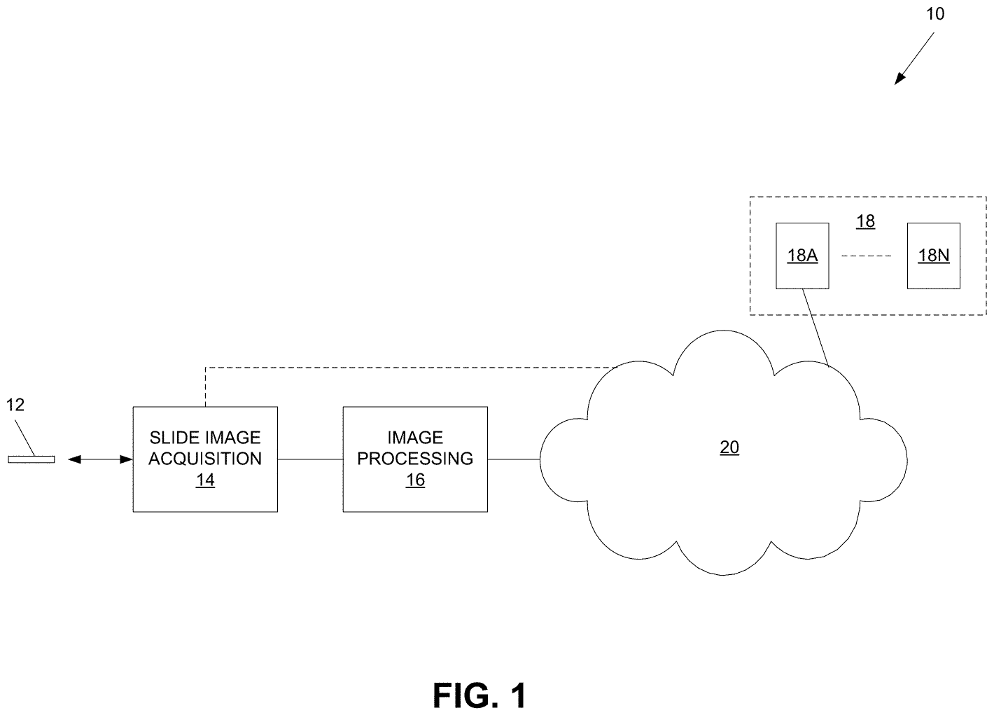

Referring now to FIG. 1, shown is an example environment 10 in which certain embodiments of a pathology imaging visualization system may be implemented. It should be appreciated by one having ordinary skill in the art in the context of the present disclosure that the environment 10 is one example among many, and that some embodiments of a pathology imaging visualization system may be used in environments with fewer, greater, and/or different components than those depicted in FIG. 1. The pathology imaging visualization system may comprise all of the devices depicted in FIG. 1 in one embodiment, or a subset of the depicted devices in some embodiments. The environment 10 comprises a plurality of devices that enable communication of information throughout one or more networks. The depicted environment 10 comprises a slide 12, a slide image acquisition system 14, an image processing system 16, and a remote processing system 18, the latter of which is communicatively coupled to the image processing system 16 and/or slide image acquisition system 14 via a network 20. In the description that follows, for the sake of ease of illustration and facilitating a ready understanding of certain embodiments of a pathology imaging visualization system, the image processing system 16 is described primarily herein as performing tissue and/or cell area detection, measurement, and visualization based on information extracted from the slide 12 (e.g., comprising a tissue sample) and provided by the slide image acquisition system 14. It is noted that the slide 12 is shown separately to merely illustrate that whole slide images are taken by the slide image acquisition system 14, and that in practice, the slide 12 is typically integrated with the slide image acquisition system 14. However, in some embodiments, the functions of detection, measurement, and visualization may be performed at each or either of the slide image acquisition system 14 (e.g., using one or more processors executing instructions), the image processing system 16, or the remote processing system 18 (e.g., using one or more processors executing instructions and residing within one or more computing devices). In some embodiments, the functions of detection, measurement, and visualization may be distributed among the slide image acquisition system 14, the image processing system 16, and the remote processing system 18. The network 20 may include one or more networks, including a wide area network (WAN), including the Internet, a metropolitan area network (MAN), one or more local area networks (LANs), a telephony network (e.g., cellular and/or landline), a wireless network, among other networks.

In one embodiment, the slide image acquisition system 14 may comprise a microscope having a motorized microscope stage for holding the slide 12, and an optical system including an objective lens. The microscope stage may be any suitable motorized stage having the necessary positioning accuracy. The motorized stage is driven under the control of a stage controller, which controls the stage in response to instructions from a computing device. The motorized stage is typically driven only in the x- and y-directions, though may be adjusted in the z-direction as well in some embodiments. Focusing of the microscope is controlled by a focus controller (e.g., piezo-electric controller) that moves (via a focusing device) the objective lens towards and away from the slide 12, along the axis of the optical system, to focus the microscope, under control of the computing device. The slide image acquisition system 14 additionally includes a digital camera, which may comprise a high resolution CCD or CMOS camera. In one embodiment, the camera includes a square array of CCD or CMOS sensors. The camera is arranged to acquire images from the microscope under control of the computing device and to provide the acquired images to the image processing system 16 for processing. Note that the slide image acquisition system 14 may be embodied in other forms, than that described above for illustration, to perform the same or similar functions, with such forms embodied, for instance, by whole slide imaging scanners that pair with slide staining techniques according to brightfield, fluorescent, and/or multispectral scanning techniques. Suitable manufacturers include the Ultra Fast Scanner, Digital Pathology Slide Scanners by Philips, Aperio Digital Pathology Slide Scanners by Leica Biosystems, Motic Whole Slide Scanners by Meyer Instruments, among others.

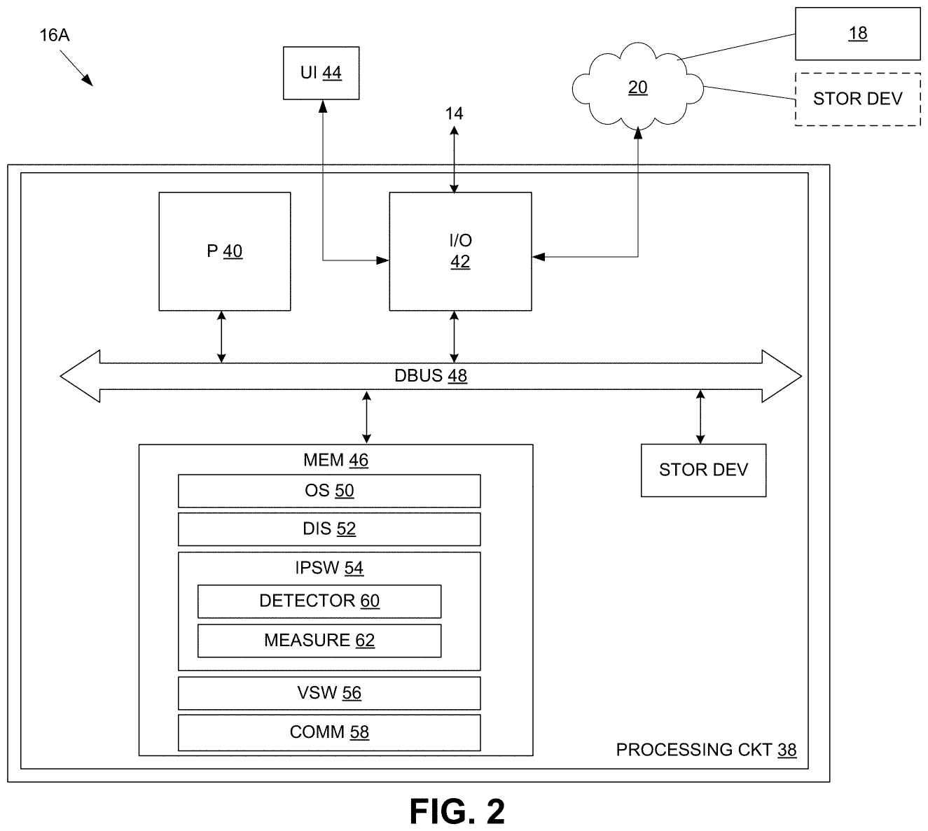

The image processing system 16 is configured to receive the digital pathology images (whole slide images) from the slide image acquisition system 14 over a wired or wireless connection and perform detection of tissue areas (e.g., tumors, stroma, adipose (fat), etc.) and measurement of the tissue areas. Further, the image processing system 16 is configured to determine the proportional value of each tissue type to all of the tissue detected in a given image (e.g., slide image), and associate each of the tissue types to a data set that corresponds to nested areas (e.g., rectangles), as described further below in association with FIG. 2. In some embodiments, the image processing system 16 is further configured to perform tissue and cell analysis (e.g., the analysis providing the proportion of a specific cell type, relative to all detected cells, in a specific tissue area). In some embodiments, the image processing system 16 comprises an integral or connected display device that presents the visualizations. In some embodiments, display devices elsewhere may be used for the visualization, including at other locations within the environment 10.

As indicated above, in some embodiments, one or more of the detection, measurement, and visualization functionality may be performed at the slide image acquisition system 14 and/or the remote processing system 18 (e.g., via communications over the network 20). In some embodiments, the remote processing system 18 may serve as storage for pathology image information that may be accessed by the image processing system 16 for processing (e.g., detection, measurement, and/or visualization). In one embodiment. processing functionality performed at the slide image acquisition system 14, image processing system 16, and the remote processing system 18 may be performed using one or more computing devices, each configured as a notebook, laptop, workstation, notepad, personal digital assistant, server device, smartphone, among other types of computing devices. In some embodiments, one or more of such computing devices may be configured as thin clients that are dedicated to rendering of visualizations based on processing performed elsewhere. In some embodiments, processing functionality of the slide image acquisition system 14, image processing system 16, and/or the remote processing system 18 may be performed using one or more discrete or integrated components, including using one or more of a digital signal processor (DSP), a graphics processing unit (GPU), a tensor processing unit (TPU), an applications specific integrated circuit (ASIC), a field programmable gate array (FPGA), among others. The slide image acquisition system 14, image processing system 16, and/or the remote processing system 18 may comprise communication functionality to enable communications over the network 20 and/or communications over other or additional networks (e.g., between the slide image acquisition system 14 and the image processing system 16 and/or the remote processing system), including functionality to enable communications via PSTN (Public Switched Telephone Networks), POTS, Integrated Services Digital Network (ISDN), Ethernet, Fiber, DSL/ADSL, Wi-Fi, among others, using TCP/IP, UDP, HTTP, DSL, among other protocols or standards.

The network 20 may include the necessary infrastructure to enable wired and/or wireless/cellular communications among the slide image acquisition system 14, image processing system 16, and/or the remote processing system 18. There are a number of different digital cellular technologies suitable for use in the network 20, including (in addition to or including those referenced above): 3G, 4G, 5G, Global System for Mobile Communications (GSM), General Packet Radio Service (GPRS), CDMAOne, CDMA2000, Evolution-Data Optimized (EV-DO), EDGE, Universal Mobile Telecommunications System (UMTS), Digital Enhanced Cordless Telecommunications (DECT), Digital AMPS (IS-136/TDMA), and Integrated Digital Enhanced Network (iDEN), among others, as well as Wireless-Fidelity (Wi-Fi), 802.11, streaming, for some example wireless technologies. As indicated above, the network 20 may include the necessary infrastructure for wired communications, including Ethernet, hybrid-fiber coaxial, copper, etc.

In one embodiment, the remote processing system 18 comprises one or more computing devices 18A through 18N, which may be configured as a single computing device or server or plural computing devices or servers (e.g., application servers, web servers, etc.), including data storage. For instance, in one embodiment, the remote processing system 18 may serve as a cloud computing environment (or other server network) for the slide image acquisition system 14 and/or image processing system 16, performing processing and/or data storage on behalf of (or in some embodiments, in addition to) the slide image acquisition system 14 and/or image processing system 16. When embodied as a cloud service or services, the remote processing system 18 may comprise an internal cloud, an external cloud, a private cloud, or a public cloud (e.g., commercial cloud). For instance, a private cloud may be implemented using a variety of cloud systems including, for example, Eucalyptus Systems, VMWare vSphere®, or Microsoft® HyperV. A public cloud may include, for example, Amazon EC2®, Amazon Web Services®, Terremark®, Savvis®, or GoGrid®. Cloud-computing resources provided by these clouds may include, for example, storage resources (e.g., Storage Area Network (SAN), Network File System (NFS), and Amazon S3®), network resources (e.g., firewall, load-balancer, and proxy server), internal private resources, external private resources, secure public resources, infrastructure-as-a-services (IaaSs), platform-as-a-services (PaaSs), or software-as-a-services (SaaSs). The cloud architecture of the remote processing system 18 may be embodied according to one of a plurality of different configurations. For instance, if configured according to MICROSOFT AZURE™, roles are provided, which are discrete scalable components built with managed code. Worker roles are for generalized development, and may perform background processing for a web role. Web roles provide a web server and listen for and respond to web requests via an HTTP (hypertext transfer protocol) or HTTPS (HTTP secure) endpoint. VM roles are instantiated according to tenant defined configurations (e.g., resources, guest operating system). Operating system and VM updates are managed by the cloud. A web role and a worker role run in a VM role, which is a virtual machine under the control of the tenant. Storage and SQL services are available to be used by the roles. As with other clouds, the hardware and software environment or platform, including scaling, load balancing, etc., are handled by the cloud.

In some embodiments, the computing devices 18A-18N of the remote processing system 18 may be configured into multiple, logically-grouped servers (run on server devices), referred to as a server farm. The computing devices 18A-18N may be geographically dispersed, administered as a single entity, or distributed among a plurality of server farms, executing one or more applications on behalf of, or processing data from, one or more of the slide image acquisition system 14 and/or image processing system 16. The computing devices 18A-18N within each farm may be heterogeneous. One or more of the computing devices 18A-18N may operate according to one type of operating system platform (e.g., WINDOWS NT, manufactured by Microsoft Corp. of Redmond, Wash.), while one or more of the computing devices 18A-18N may operate according to another type of operating system platform (e.g., Unix or Linux). The computing devices 18A-18N may be logically grouped as a farm that may be interconnected using a wide-area network (WAN) connection or medium-area network (MAN) connection. The computing devices 18A-18N may each be referred to as, and operate according to, a file server device, application server device, web server device, proxy server device, or gateway server device.

The remote processing system 18 may maintain one or more data structures (e.g., expert data structures) and/or receive data collected via one or more of the slide image acquisition system 14 and/or image processing system 16 and store the received data in one or more data structures and/or process the information, and communicate the information back to the slide image acquisition system 14 and/or image processing system 16 or present information to a user interface (e.g., serving a web server function, or rendering information to a local display device).

Note that in some embodiments, processing functionality of the image processing system 16 may involve plural computing devices used as an edge or local computing network for processing the digital pathology tissues.

Cooperation between the slide image acquisition system 14, image processing system 16, and the remote processing system 18 may be facilitated (or enabled) through the use of one or more application programming interfaces (APIs) that may define one or more parameters that are passed between a calling application and other software code such as an operating system, library routine, and/or function that provides a service, that provides data, or that performs an operation or a computation. The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer employs to access functions supporting the API. In some implementations, an API call may report to an application the capabilities of a device running the application, including input capability, output capability, processing capability, power capability, and communications capability.

Referring now to FIG. 2, shown is an example computing device 16A that is configured to implement certain functionality of an embodiment of a pathology imaging visualization system. In particular, the example computing device 16A represents one illustrative embodiment of an image processing system 16 configured as a computing device, which may be configured as an application server, computer, among other computing devices. In some embodiments, the image processing system 16 may be embodied in other forms, including as one or more of a DSP, GPU, TPU, ASIC, FPGA, among other devices that can execute instructions (firmware, software, microcode, etc.). Though described as follows in the context of the computing device 16A depicted in FIG. 2, it should be appreciated by one having ordinary skill in the art that the functionality of a pathology visualization system is not so limited and may be embodied in one or more other types of devices. Hereinafter, the computing device 16A and image processing system 16 are terms that are used interchangeably. One having ordinary skill in the art should appreciate in the context of the present disclosure that the example computing device 16A is merely illustrative of one embodiment, and that some embodiments of computing devices may comprise fewer or additional components, and/or some of the functionality associated with the various components depicted in FIG. 2 may be combined, or further distributed among additional modules or computing devices, in some embodiments. It should be appreciated that certain well-known components of computers are omitted here to avoid obfuscating relevant features of the computing device 16A. In one embodiment, the computing device 16A comprises a processing circuit 38 (PROCESSING CKT) that comprises one or more processors (one shown), such as processor 40 (P), input/output (I/O) interface(s) 42 (I/O), one or more user interfaces (UI) 44, which may include one or more of a keyboard, mouse, microphone, speaker, tactile device (e.g., comprising a vibratory motor), etc., and memory 46 (MEM), all coupled to one or more data busses, such as data bus 48 (DBUS). In some embodiments, the user interfaces may be coupled directly to the data bus 48.

The memory 46 may include any one or a combination of volatile memory elements (e.g., random-access memory RAM, such as DRAM, and SRAM, etc.) and nonvolatile memory elements (e.g., ROM, Flash, solid state, EPROM, EEPROM, hard drive, tape, CDROM, etc.). The memory 46 may store a native operating system, one or more native applications, emulation systems, or emulated applications for any of a variety of operating systems and/or emulated hardware platforms, emulated operating systems, etc. In some embodiments, a separate storage device (STOR DEV) may be coupled to the data bus 48, or as a network or external connected device (or devices, as shown in phantom in FIG. 2) via the I/O interfaces 42 and the network 20. The storage device may be embodied as persistent memory (e.g., optical, magnetic, and/or semiconductor memory and associated drives), and in some embodiments, may be used to store, all or in part, data depicted as stored in memory 46. In some embodiments, network connected storage devices may include personal health record data storage, electronic health data record storage, and/or data storage for other institutions (e.g., research institutions, including cohort data).

In the embodiment depicted in FIG. 2, the memory 46 comprises an operating system 50 (OS), digital image storage (DIS) 52, and application software that includes image processing software (IPSW) 54 and visualization software (VSW) 56. Memory 46 further comprises communications software (COMM) 58, which may include middleware (e.g., browser software), APIs, and/or software to enable wired and/or wireless communications over one or more networks. Note that the memory 46 (or storage device(s)) may also be referred to herein as a non-transitory, computer-readable (storage) medium. Though depicted as software in FIG. 2, the application software that includes the image processing software 54, visualization software 56, and communications software 58 may be embodied as other types of instructions, including firmware or microcode, depending on the particular implementation of the image processing system 16.

In one embodiment, the digital image storage 52 comprises a data structure or digital image library of digital pathology images acquired from the slide image acquisition system 14 (e.g., based on image capture of tissue samples on slide 12). In some embodiments, raw digital pathology images may be communicated from the slide image acquisition system (directly or indirectly via a suitable gateway, such as a wireless or cable modem) to storage devices associated with the remote processing system 18, and later accessed prior to processing by the computing device 16A.

The image processing software 54 comprises executable code (instructions) that, when executed by the processor 40 (or processors), configures the processor 40 to receive or access each of the digital pathology images, detect tissue areas, and measure each of the tissue areas. In one embodiment, the image processing software 54 comprises a tissue area detector module (detector) 60 (also referred to herein as simply tissue detector module 60 or detector module 60) and a measurement module (measure) 62, which each comprise instructions to configure the processor 40 to perform detection of the tissue area (and cellular content) for each slide image and measure each of the tissue and/or cell areas for each slide image. In one embodiment, the tissue area detector module 60 is configured to perform standard segmentation analysis followed by application of supervised learning methods or unsupervised learning methods for tissue and/or cell classification (see e.g., Machine Learning Methods for Histopathological Image Analysis, Komura et al., Cornput. Struct. Biotechnol. J. 16 (2018), 34-42, which may be applied to tissue and cell detection). In one embodiment, the tissue area detector module 60 outputs tissue and cell classifications according to different classes. For the tissue type detection, the detector module output may be represented by a set of images patches, derived from the whole-slide image, classified according to their content. For cell detection, the output may be represented by the coordinates of the detected and classified cell in image pixel space (e.g., cell, coordinate x, coordinate y, class, such as, #1, 5120, 10304, tumor, #2, 5123, 100308, lymphocyte, etc.). In one embodiment, the output is stored in files that follow a prescribed ontology or schema. In one embodiment, the tissue area detector module 60 generates polygon instances characterized by boundaries of the tissue regions on the whole-slide image. Additional information on tissue segmentation and/or cell/nuclei detection is described in Breast Cancer Histopathology Image Analysis: A Review, Veta et al., IEEE Transactions on Biomedical Engineering (Volume 61, Issue 5, May 2014) and Deep Learning for Digital Pathology Image Analysis: A Comprehensive Tutorial With Selected Use Cases, Janowczyk et al., J Pathol Inform, 2016.

The measurement module 62 is configured to receive as input the data or information generated by the tissue area detector module 60, including the tissue classification and/or a cell detection list. In one embodiment, the measurement module 62 receives the polygonal instances generated by the tissue area detector module 60. Once a polygon is generated, the area of a particular region may be derived using the image resolution information (e.g., 0.25 micrometer/pixel, such as received from the slide image acquisition system 14 and/or pre-programmed) to generate such information. In one embodiment, the measurement module 62 performs cell measurement according to a counting process that involves calculation of a number of cell classes inside a specific tissue region (e.g., inside polygon boundary coordinates). In one example process performed by the measurement module 62, the measurement module 62 (1) calculates the area of the detected tissue, (2) calculates the areas of the detected tissue types, (3) calculates the percentages of (2) with respect to (1), (4) calculates the number of detected cells per type in each region by using the list provided by the tissue area detector module 60 (e.g., coordinates and class labels are used in (4)), and (5) calculates the percentage of (4) with respect to all cells detected in the region.

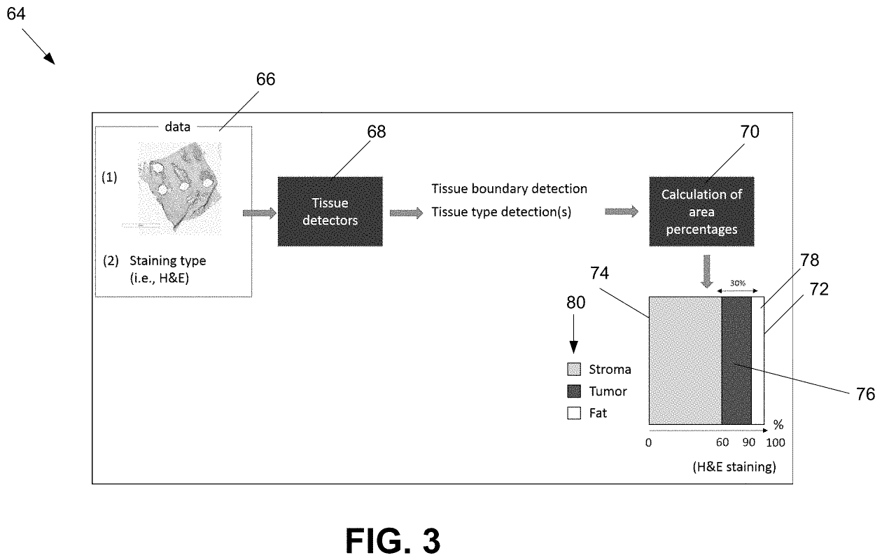

The visualization software 56 comprises executable code (instructions) that, when executed by the processor 40 (or processors), configures the processor 40 to visually represent each of the tissue areas as a proportion of all of the tissue areas in a whole slide image using one or more respective nested rectangles that are located entirely within a single rectangle, the nested rectangles proportional in area to the respective proportions of the tissue areas of the slide image. Note that rectangles are used herein as one example visual area representations among other types of visual area representations. Note that in some embodiments, at the risk of compromising the intuitiveness of the visualization, the single rectangle may be omitted. For instance, if the tissue area detector module 60 identifies tumor, stroma, and fat tissue perfectly but is incapable of identifying the remaining 10% of the tissue, a visualization with the single rectangle allows for a nested rectangle for the unidentified tissue under the category of other (e.g., as an empty rectangle) while still providing insight regarding the total tissue amount (whereas without the single rectangle, such information is missing or may be less insightful to obtain). In general, the visualization software employs a specific visualization of tree-maps to encode the image analysis output from the image processing software 54 to display digital pathology images on the user interface 44 (e.g., on a GUI presented on the user interface 44) and/or to communicate the visualization to other display devices. In one embodiment, the visualization software 56 uses a rectangular shape S to represent a singular, digital pathology image or the digital slide (image). This rectangle represents the detected tissue region (100%) on the digital pathology image. For instance, and referring also to the diagram 64 of FIG. 3 for illustration, information or data (66) comprising the digital pathology image for a single slide is received by the image processing software 54, where the tissue areas (e.g., tumor, stroma, fat, etc.) are detected (68) by the tissue area detector module 60 (e.g., tissue detectors in FIG. 3). Similar functionality applies to cell analysis, with emphasis below on tissue analysis. The detector module 60 determines tissue boundaries and tissue types, and outputs this information to the measurement module 62 to calculate (70) the tissue areas and percentages or proportions of the tissue areas relative to the aggregate of all of the tissue areas detected in the digital pathology image (slide). The measurement module 62 provides this calculated information to the visualization software 56, which renders a visual representation (72) of the tissue content percentages relative to the entire tissue detected and measured (100%). That is, the visualization software 56 takes the tissue/cell hierarchy and the respective measurements and generates a tree map visualization. In this example, the visualization or visual representation 72 comprises three nested rectangles 74, 76, and 78 proportioned according to the area of the respective tissue type each represents, these rectangles 74, 76, and 78 all nested within a single rectangle. A scale is shown for illustration (though in practice, not necessarily part of the visualization), indicating the different percentages of the different tissue types. For instance, the visualization 72 shows tissue type of stroma, represented by nested rectangle 74, contributing 60% of the entire tissue content, the tumor, represented by nested rectangle 76, contributing 30%, and fat, represented by nested rectangle 78, contributing 10%. Thus, areas are distributed by following a horizontal layout (e.g., left to right), from a 0% tissue area to 100%.

In some embodiments, each nested rectangle 74, 76, and 78 is colored by following a color scale that should resemble the biological visual aspect of the tissue types of the digital pathology images (according to the staining). For instance, by employing a dark blue/violet to the tumor areas, a user (e.g., a pathologist) can immediately benefit from the tree-map visualization to understand the tumor content of one or multiple slides. Further illustrating the color scheme is an annotation 80 (e.g., a legend), which in some embodiments, may be included as part of the visualization 72. In this example, the annotation 80 comprises three, visually distinguishable, geometric symbols (e.g., boxes), corresponding respectively to the three different tissue types (and hence, three nested rectangles 74, 76, and 78) from this slide image. In this example, the boxes of the annotation 80 are visually distinguished by color (e.g., of the staining used in the slide imaging for the different tissue types), with the corresponding colors used for the nested rectangles 74, 76, and 78. That is, the colored annotation box for stroma is of the same color as the nested rectangle 74, signifying that the rectangle 74 corresponds to or is associated with the stroma tissue type. Similarly, the darker colored annotation box for tumor is of the same color as nested rectangle 76, signifying that the rectangle 76 corresponds to, or is associated with, the tumor tissue type. Similarly, the lighted colored annotation box for fat is of the same color as nested rectangle 78, signifying that the rectangle 78 corresponds to, or is associated with, the fat tissue type. In some embodiments, the annotations may represent cell types and/or other type of tissue and/or cell information.

As illustrated in FIG. 3, tissue detectors (the tissue detector module 60) detect and label the tissue types on the digital pathology image, and the measurement module 62 calculates area percentages (e.g., tissue area percentages, cell percentages), which is input to the visualization software 56 to generate the tree map. That is, the visualization software 56 renders a display of one or more nested rectangles s in S. Note that in some embodiments, the data 66 includes the staining type (e.g., H&E) used for the slide imaging, which is used for distinguishing the nested rectangles and generating the annotation (legend) 80.

In general, the visualization software 56 provides a graphical interface containing a set of graphical representations of a digital slide which content varies as a function of certain clinical parameters determined by the computing device 16. As described above, the tumor proportion may be computed and represented with a corresponding area of a predefined color inside the whole slide area represented. In a sense, one benefit of certain embodiments of a pathology imaging visualization system is not one of a presentation of information per se. The slide representations emulate a rough representation of each slide and its respective tissue (e.g., tumor, fat, stroma) and/or cell percentages as it passes through processing. In effect, certain embodiments of a pathology imaging visualization system solve the problems or challenges involved with providing a visualization of digital pathology images for GUI. The visualization encodes image analysis information relevant to the pathology domain, and it can be flexible and adaptable to the color scheme of the image (due to staining) and interactions that may be provided.

Explaining the image processing software 54 and visualization software 56 further, the data (66) comprises, in one embodiment, a digital pathology image and the specification of the staining used to obtain the final glass slide image. The image processing software 54 processes the digital pathology image by image analysis detectors (of the tissue detector module 60) capable of detecting the tissue (or cellular) content of the image (and perform measurements (e.g., the measurement module 62)) that map it to a hierarchical dataset in the following structure:

Root:
Label: Tissue boundaries
Value: 100%
Children:
   Children 1:
      Label: Tissue type 1
      Value: Value[Tissue type 1]
      Children: [ . . . ]
   Children 2:
      Label: Tissue type 2
      Value: Value[Tissue type 2]
      Children: [ . . . ]
   . . .
   Children N:
      Label: Tissue type N
      Value: Value[Tissue type N]
      Children: [ . . . ]

In one embodiment, children 1 may include tissue types (e.g., tumor, stroma, fat, etc.), and children 2 may include cell types (e.g., tumor cells, immune cells, epithelial cells, etc.). The particular breakdown depends on the tissue type (e.g., breast, liver, lung, etc.) and the capabilities of the detection system and the way data is generated and specified. The hierarchical dataset is data used by the visualization software 56 to generate a 2D visualization and nested rectangles. In some embodiments, information about the digital image staining may be used to retrieve the corresponding color scale to apply to the visualization. Several color scales matching the biological content of digital pathology images can be generated and stored. Such color scales may provide an advantage to the user for recognition of specific histopathology components, which are common in the pathology domain. In some embodiments, the dataset may be configured for visualization of cell type proportions. For instance, children 1 may be configured as follows: label: cell type 1, value: Value[cell type 1], children: none (e.g., cells, since in this example at the bottom of the hierarchy, may not include something else).

Note that in some embodiments, functionality of the image processing software 54 and visualization software 56, and/or tissue detector module 60 and measurement module 62, may be combined into a single module, or further distributed among more modules. Functionality of the image processing software 54 and the visualization software 56 may reside at each of the slide image acquisition system 14, image processing system 16 (e.g., computing device 16A), and the remote processing system 18, elsewhere than the computing device 16A (e.g., slide image acquisition system 14 or the remote processing system 18), or distributed among the slide image acquisition system 14, image processing system 16 (e.g., computing device 16A), and the remote processing system 18.

The communications software 58 operating in conjunction with the I/O interfaces 42 (collectively referred to as a communications interface) comprises functionality to communicate with other devices of the network, including devices of the slide image acquisition system 14 and/or the remote processing system 18, and in some embodiments may include connectivity logic for communications with an Ethereum network, Ethernet network, hybrid-fiber coaxial (HFC) network, among other wired and/or wireless networks. In some embodiments, the communications software 58 comprises middleware that includes a web server, web browser, among other software or firmware.

Execution of the image processing software 54 (including associated modules 60, 62), visualization software 56, and communications software 58 may be implemented by the processor 40 (or processors) under the management and/or control of the operating system 50. The processor 40 may be embodied as a custom-made or commercially available processor, a central processing unit (CPU) or an auxiliary processor among several processors, a semiconductor based microprocessor (in the form of a microchip), a macroprocessor, one or more application specific integrated circuits (ASICs), a plurality of suitably configured digital logic gates, and/or other well-known electrical configurations comprising discrete elements both individually and in various combinations to coordinate the overall operation of the computing device 16A.

The I/O interfaces 42 comprise hardware and/or software to provide one or more interfaces to devices coupled to one or more networks, including network 20, as well as to other devices, such as the user interface 44 or the slide image acquisition system 14. In other words, the I/O interfaces 42 may comprise any number of interfaces for the input and output of signals (e.g., analog or digital data) for conveyance of information (e.g., data) over various networks and according to various protocols and/or standards.

The user interfaces 44 may include a keyboard, mouse, microphone, immersive head set, display device, gesture or user motion detecting/recognition interface, speech, video, or image-based detection/recognition interfaces, etc., which enable input and/or output by a user. In some embodiments, the user interface 44 may be configured for visual rendering according to augmented or virtual reality. In some embodiments, the user interface 44 may be omitted and located elsewhere.

When certain embodiments of the computing device 16A are implemented at least in part with software (including firmware), as depicted in FIG. 2, it should be noted that the software (e.g., image processing software 54 (including associated modules 60, 62), visualization software 56, and communications software 58) can be stored on a variety of non-transitory computer-readable (storage) medium for use by, or in connection with, a variety of computer-related systems or computer-implemented methods. In the context of this document, a computer-readable storage medium may comprise an electronic, magnetic, optical, or other physical device or apparatus that may contain or store a computer program (e.g., executable code or instructions) for use by or in connection with a computer-related system or method. The software may be embedded in a variety of computer-readable storage mediums for use by, or in connection with, an instruction execution system, apparatus, or device, such as a computer-based system, processor-containing system, or other system that can fetch the instructions from the instruction execution system, apparatus, or device and execute the instructions.

When certain embodiments of the computing device 16A are implemented at least in part with hardware, such functionality may be implemented with any or a combination of the following technologies, which are all well-known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), relays, contactors, etc.

It is noted that in some embodiments, the architecture and corresponding functionality described for the computing device 16A may apply in whole or in part to computing devices associated with the slide image acquisition system 14 and/or the remote processing system 18.

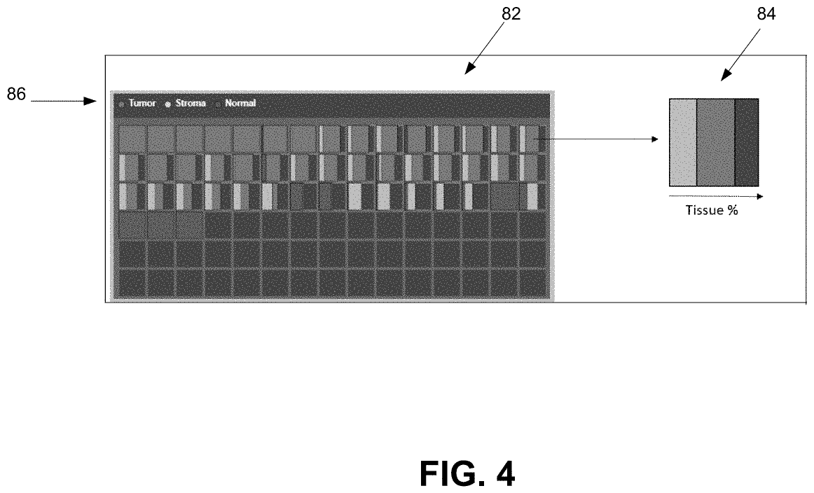

Having described an example environment and processing architecture in which certain embodiments of a pathology imaging visualization system may be implemented, attention is now directed to FIG. 4, which illustrates an example visualization 82 for plural digital pathology images. That is, the visualization 82 may be generated by the visualization software 56 in the manner described above in association with FIG. 2, and comprises a digital pathology image view of plural slides. Each slide or digital pathology image is visually represented by a respective slide visualization 84, which comprises in this example plural nested rectangles within a single rectangle, each nested rectangle representing a tissue type and proportional content (tissue proportions, cell proportions) for that slide, as similarly described for visualization 72 of FIG. 3. Also, in the depicted embodiment, the visualization 82 comprises an annotation 86 that signifies the tissue type each color represents (e.g., for tumor, stroma, and normal). Note that in some embodiments, though the visualization 82 is presented as a stand-alone graphical user interface, in some embodiments, the visualization 82 may be presented in conjunction with other information, including metadata associated with each of the rectangles.

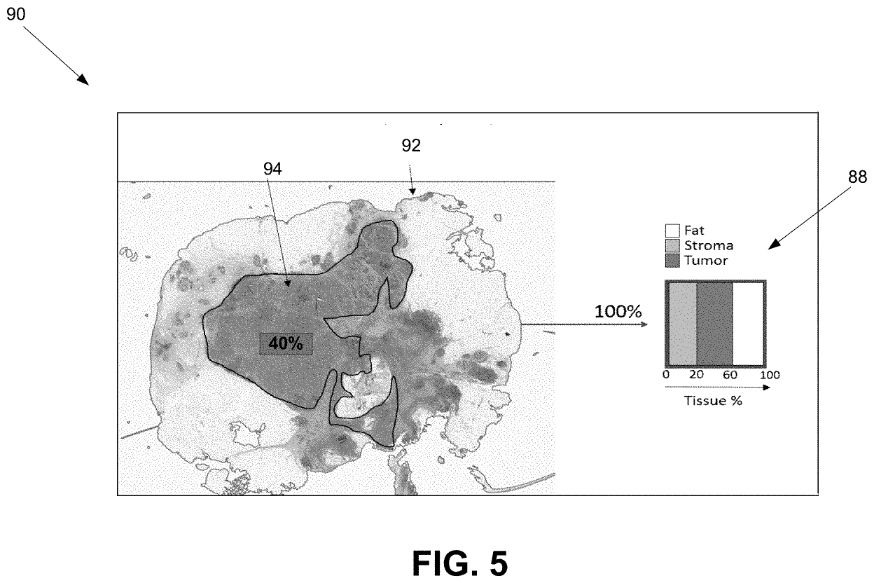

FIG. 5 conceptually illustrates how an example visualization 88 conveys information about tissues and their respective types for a digital pathology image 90. That is, FIG. 5 provides insight on how the tree-map visualization 88 is generated from the image analysis output corresponding to the digital pathology image 90 (whole slide image). The percentages of the tissue types are computed after the image analysis. The tissue boundaries (red) provide the boundaries within which the 100% is encoded with the rectangle of the tree-map visualization. Shown in the image 90 is detected area 92, including detected tumor area 94. In general, FIG. 5 is intended to convey that shape S described above represents 100% of tissue detected by the tissue detector module 60 (FIG. 2), and that the measurement module 62 (FIG. 2) uses this 100% detected tissue to calculate the other portions (other nested rectangles, s). The relation between detected tissue and tissue types is encoded in the visualization 88. Certain embodiments of a pathology imaging visualization system enable the user to see the respective tissue type on the whole slide image presented on the tree map visualization and vice versa. Note that a similar functionality described for the example visualizations in the figures is applicable to cell analysis.

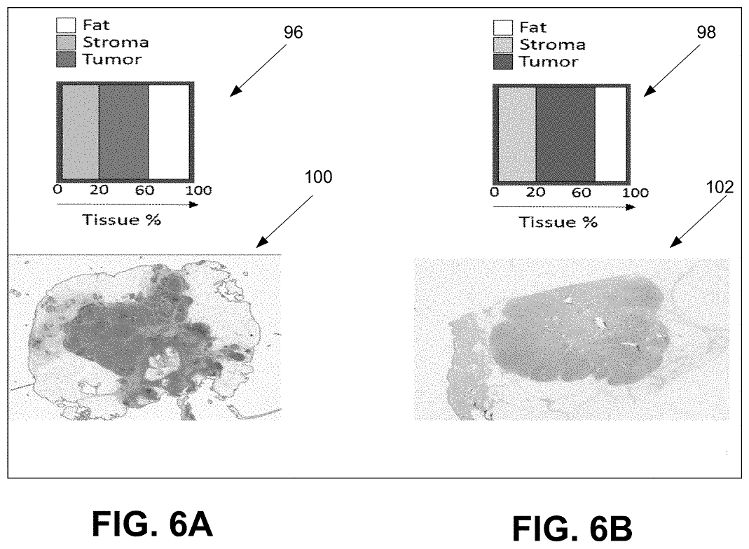

FIGS. 6A-6B are schematic diagrams that illustrate example visualizations 96 and 98, respectively, based on different staining colors. That is, visualization 96 is generated using a different type of color scheme to resemble biological structures in the associated digital pathology image 100 than that used for generating visualization 98 corresponding to digital pathology image 102, each of which use a different type of staining. As indicated above, information about the staining may be included as part of data 66 (FIG. 3) received by the image processing software 54 (FIG. 2). In this example, the visualization 96 is generated based on a color scheme associated with the Hematoxylin and Eosin (H&E) staining used for the image 100, whereas the visualization 98 is generated based on a color scheme associated with the Immunohistochemistry staining used for the image 102. The difference in stains is manifested in a difference in color scheme for each visualization 96, 98. In general, plural color schemes may be employed in some embodiments according to the staining technique applied to the tissue slides. In some embodiments, though tissue type and staining technique are visually represented by visualizations 96, 98, in some embodiments, other and/or additional characteristics may be visually represented including, for instance, mitotic figures, nuclear density, grading, among other tissue and/or cellular information.

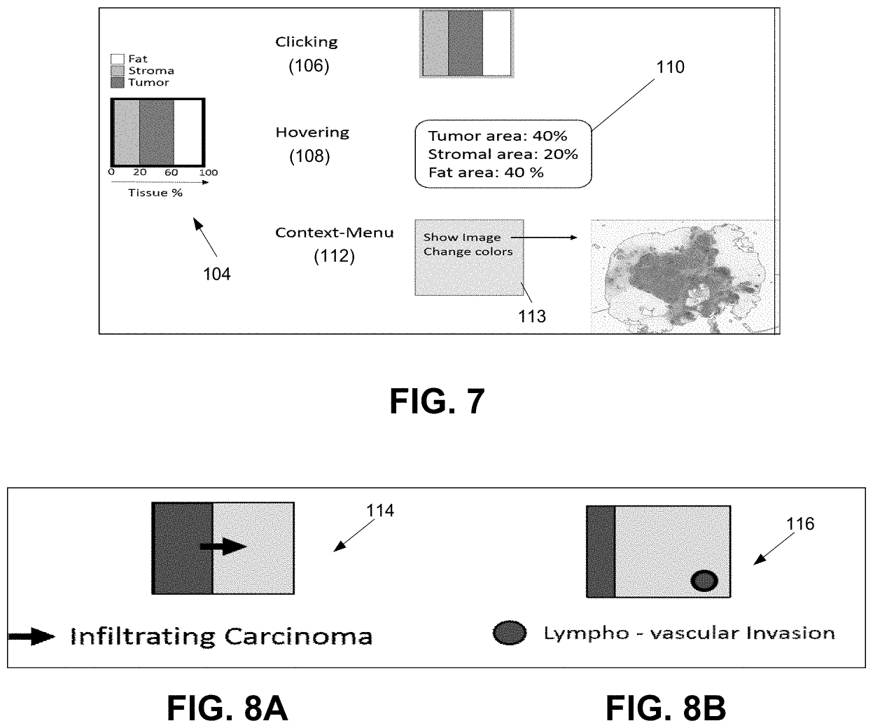

FIG. 7 is a schematic diagram that conceptually illustrates interactive functionality based on a type of user input enabled by an example pathology imaging visualization system. An example visualization 104 is shown on the left hand side of the diagram, which illustrates nested rectangles within a single rectangle as described above. At the top, clicking user input 106 is represented, where the functionality involved with a clicking input (e.g., a single mouse click or a single touch on a touch-type display) on a nested rectangle shows nested children in the hierarchy dataset. In the middle of the diagram, a hovering user input 108 is shown, wherein, for instance, dragging a mouse over an area of the visualization 104 prompts the presentation of a tooltip 110, here showing, in text, what the visualization 104 represents (e.g., tumor area: 40%, stromal area: 20%, fat area: 40%). In the lower part of the diagram, shown is a context-menu user input 112, wherein an input graphic 113 enables a user to show the original digital pathology image corresponding to the visualization 104, or an option to change colors. For instance, the input graphic 113 may comprise a dialog box that can be opened, for instance, with a right-click on the tree map visualization 104, and that provides a user with more options to interact/view the visualization of the tree map 104. As one example, the show image selection may display the corresponding thumbnail or the full extent whole slide image in another window.

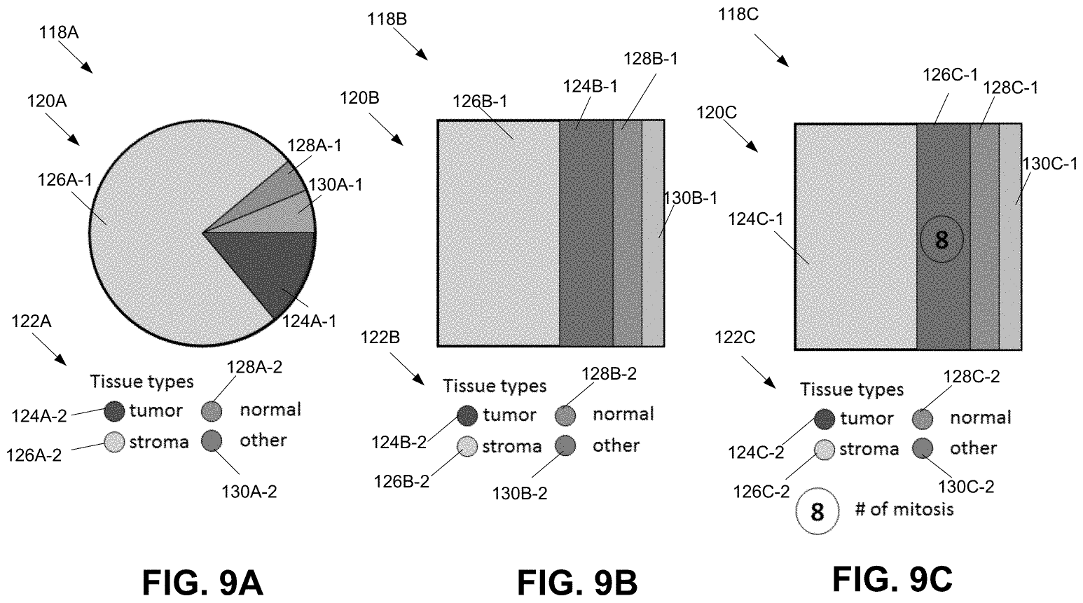

FIGS. 8A-8B are schematic diagrams that illustrate example symbols used to visually represent various tissue content information for an example pathology imaging visualization system. That is, tissue type rectangles (or other types of areas) can serve to encode more information obtained by the image analysis. For instance, if tumor cells are present in the stromal tissue, the tumor is considered an infiltrating cancer. In FIG. 8A, this is shown with a visualization 114 with an arrow symbol that goes from the tumor area (rectangle) to the stromal area. If lympho-vascular invasion is detected in a region, this can be shown with a visualization 116 with a circle symbol as shown in FIG. 8B. This feature may add to the topological content on the digital images and may help the pathologists to identify/assess the presence of specific components. It should be appreciated by one having ordinary skill in the art, in the context of the present disclosure, that the symbols used in these FIGS. 8A-8B are merely illustrative, and that in some embodiments, symbols of different sizes, shapes/types (e.g., polygons, text, numbers, alphanumerics, or other types of symbols), and/or location may be used. In addition, other mechanisms for visually distinguishing the symbols (and providing corresponding, distinguishable visual representations) may be used, including through the use of differences in patterns, color/translucence, shading, etc.). In some embodiments, the visualizations 114 and 116 may have annotations (e.g., legends), as respectively shown beneath the rectangles and symbols in FIGS. 8A-8B, which shows that the arrow symbol corresponds to an infiltrating carcinoma and the circle symbol (e.g., a colored symbol, or other fills, including no fill) corresponding to a lympho-vascular invasion.

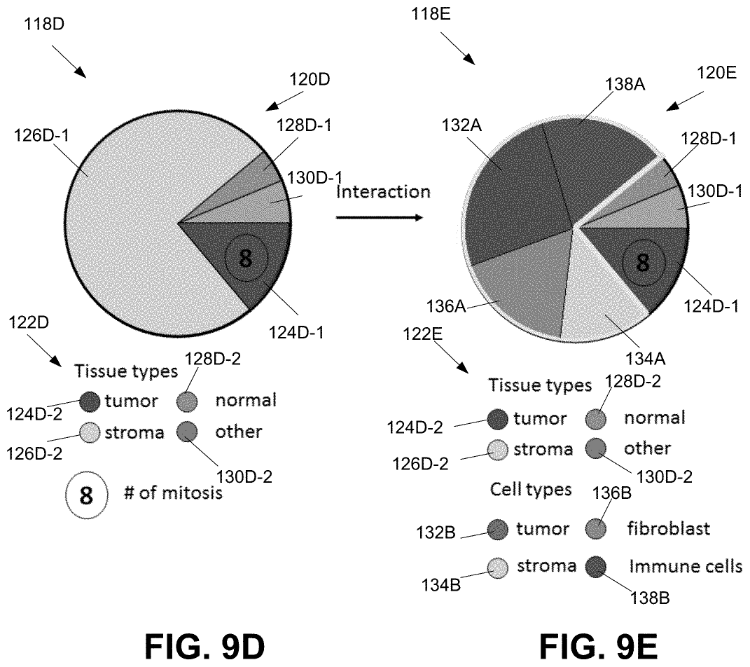

FIGS. 9A-9E are schematic diagrams that illustrate different area representations for example visualizations that may be used for an example pathology imaging visualization system. As similarly discussed above, these visualizations may be based on analysis of a whole slide image where a detector (or in some embodiments, based on manual detection and labelling) has identified the sample as having a tissue area, stroma area, normal area, and other. As explained above, certain embodiments of a pathology imaging visualization system measure these areas and provide a visualization, the visualization including nested, interactive areas representing the tissue or cell areas in corresponding proportion. In some embodiments, the nested areas that correspond to the whole slide image areas may be colored according to the corresponding staining mechanism used in the whole slide image for the respective tissue areas. In some embodiments, an annotation (e.g., legend) may also be presented, as described above. Referring in general to FIGS. 9A-9E, shown are example visualizations 118 (e.g., 118A-118E), each with a geometric shape 120 (e.g., 120A-120E) and an associated annotation or legend 122 (e.g., 122A-122E). Referring in particular to FIG. 9A, the visualization 118A comprises the geometric shape 120A and the corresponding annotation 122A. The geometric shape 120A is embodied as a circle, with nested arcs or pie-segments (e.g., pie chart). The single circle represents 100% of the whole slide image, and the nested arcs represent a proportion of the tissue type within the whole slide image. In this example, the tissue type of tumor is shown as arc 124A-1, with a corresponding annotation of 124A-2. The tissue type of stroma is shown as arc 126A-1, with a corresponding annotation of 126A-2. The tissue type of normal is shown as arc 128A-1, with a corresponding annotation of 128A-2. The balance of the tissue type(s) identified as other corresponds to arc 130A-1, with a corresponding annotation of 130A-2. As noted the proportion of the tissue type from the whole slide image is represented by a proportional arc within the single circle, and the annotation indicates the tissue type (which may comprise a color corresponding to the staining scheme, or in some embodiments, other methods of providing visual distinction among the arcs).

Referring to FIG. 9B, shown is a visualization 118B that comprises a geometric shape 120B and a corresponding annotation 122B. In this example, the geometric shape 120B comprises a single rectangle (representing 100% of the whole slide image) with nested rectangles (corresponding to tissue areas) residing (completely) within the single rectangle. In this example, the tissue type of tumor is shown as rectangle 124B-1, with a corresponding annotation of 124B-2. The tissue type of stroma is shown as rectangle 126B-1, with a corresponding annotation of 126B-2. The tissue type of normal is shown as rectangle 128B-1, with a corresponding annotation of 128B-2. The balance of the tissue type(s) identified as other corresponds to rectangle 130B-1, with a corresponding annotation of 130B-2. As noted the proportion of the tissue type from the whole slide image is represented by a proportional rectangle within the single rectangle, and the annotation indicates the tissue type as similarly described above.

As similarly described above for visualizations depicted in FIGS. 3-8B, the nested areas are interactive (enable further information based on selection), and may include in some embodiments one or more symbols to learn of additional information. Referring to FIG. 9C and as similarly described in conjunction with FIGS. 8A-8B, one or more of the nested areas (e.g., rectangles in this example) may comprise a symbol inserted in or around the rectangle that indicates additional information. For instance, in FIG. 9C (as similarly described for FIG. 9B), the visualization 118C comprises a geometric shape 120C that includes the tissue type of tumor shown as rectangle 124C-1, with a corresponding annotation of 124C-2. The tissue type of stroma is shown as rectangle 126C-1, with a corresponding annotation of 126C-2. The tissue type of normal is shown as rectangle 128C-1, with a corresponding annotation of 128C-2. The balance of the tissue type(s) identified as other corresponds to rectangle 130C-1, with a corresponding annotation of 130C-2. In this example, a symbol, shown as a non-limiting illustration with a numeral 8 residing within an un-filled circle, is located within the nested rectangle 126C-1 representing the tumor tissue type, where the number eight (8) is intended to convey the number of mitotic cells in this particular region (e.g., eight (8)). It should be appreciated that other types of symbols may be used to represent this cell information or other cell information in this rectangle 126C-1 or other rectangles. The annotation 122C, in one embodiment, identifies the symbol and its meaning.

Continuing the example of embedded symbols, reference is now made too FIG. 9D, which shows a visualization 118D having a geometric shape 120D of a single circle with nested arcs 124D-1 (tumor), 126D-1 (stroma), 128D-1 (normal), and 130D-1 (other), as identified also in annotation 122D (e.g., tumor identifier/icon 124D-2, stroma 126D-2, normal 128D-2, and other 130D-2). A symbol like that indicated for FIG. 9C (e.g., number 8 within an un-filled (e.g., taking on as background the color of the area 124D-1) circle) is embedded in arc 124D-1, representing the number of mitotic cells in this particular region. A corresponding legend is shown in the annotation 122D.

As explained above, the nested areas may be interactive. FIG. 9E illustrates on example, where user selection of the stroma area 126D-1 in FIG. 9D results in a rendering of tissue areas 124D-1, 130D-1, and 128D-1, as similarly described in FIG. 9D, in FIG. 9E, and with an exploded or refined view of the stroma area 126D-1 (FIG. 9D). In this example, the stroma area comprises proportional arcs (e.g., proportional to detected tissue/cell types) for tissue and/or cell types of tumor 132A, stroma 134A, fibroblast 136A, and immune cells 138A, each of which are further identified in annotations 122E via identifiers/icons for tumor 132B, stroma 134B, fibroblast 136B, and immune cells 138B, respectively. In one embodiment, the annotation 122E comprises the tissue and cell types, and in some embodiments, only the nested areas for the desired tissue under further evaluation and refinement (the stroma area 126D-1 in this example) have annotations shown.

In some embodiments, the symbols provide a function of presenting further information when selected (e.g., to convey the corresponding information), and in some embodiments, the symbols are sufficient to convey the information without selection (e.g., do not provide any further information when selected), and in some embodiments, a mix of each type are presented. Other mechanisms of conveying information than the symbols used herein as examples, including colored sub-regions (e.g., rectangles or other polygons), such as to convey the presence of tumor cells, lymphocytes, etc. In general, the visualizations comprise interactive areas that are proportional to the measured tissue and/or cell characteristics, and in some embodiments, include symbols to depict additional information may be in the form of further shapes, characters, symbols, colors, among other techniques for conveyance of information and/or differences.

The representation of the digital slides according to certain embodiments of a pathology imaging visualization system enables clear and direct information about its clinical content in a complex interface environment. Note that the visualizations of the pathology imaging visualization system may similarly be applied in other GUI applications, including for use in diagnostic software used by pathologists, lab software after glass slides are digitized and processed by an image analysis system, etc.

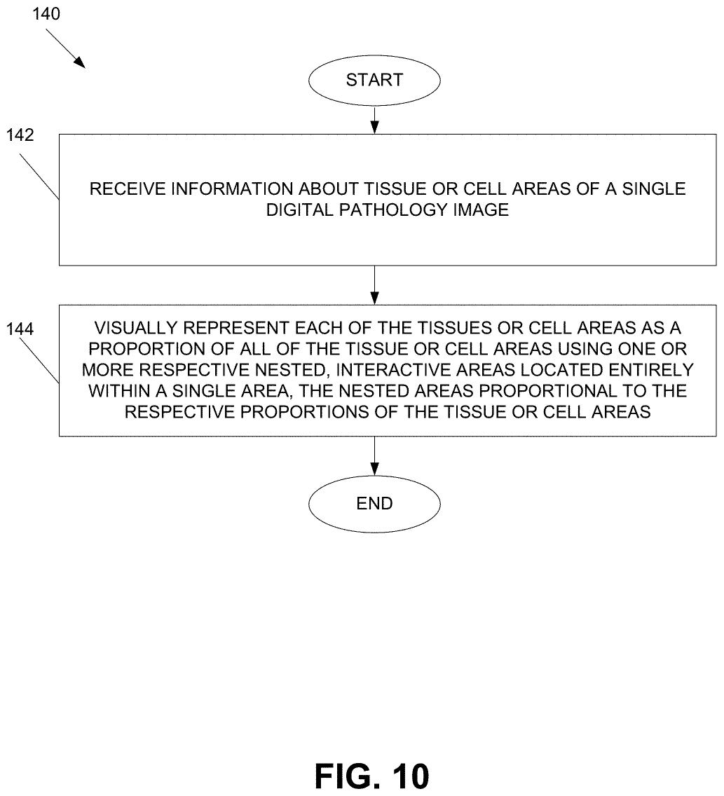

Having described certain embodiments of an example pathology imaging system, it should be appreciated that one embodiment of an example pathology imaging visualization method, performed by one or more processors of one or more computing devices, is depicted in FIG. 10 and denoted as method 140, which is shown bounded by a start and end. The method 140 comprises receiving information about tissue or cell areas of a single digital pathology image (142); and visually representing each of the tissue or cell areas as a proportion of all of the tissue areas using one or more respective nested, interactive areas located entirely within a single area, the nested area proportional to the respective proportions of the tissue or cell areas (144).

Any process descriptions or blocks in flow diagrams should be understood as representing modules, segments, or portions of code which include one or more executable instructions for implementing specific logical functions or steps in the process, and alternate implementations are included within the scope of the embodiments in which functions may be executed out of order from that shown or discussed, including substantially concurrently or in reverse order, depending on the functionality involved, as would be understood by those reasonably skilled in the art of the present disclosure. In some embodiments, one or more steps may be omitted, or further steps may be added.

Certain embodiments of a pathology imaging system aim at providing visualization that encodes image analysis outputs to represent digital pathology images on a GUI in an alternative and more illustrative way to users (e.g., pathologists, histo-technicians, researchers, etc.).

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

Note that various combinations of the disclosed embodiments may be used, and hence reference to an embodiment or one embodiment is not meant to exclude features from that embodiment from use with features from other embodiments. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical medium or solid-state medium supplied together with or as part of other hardware, but may also be distributed in other forms. Any reference signs in the claims should be not construed as limiting the scope.

At least the following is claimed:

1. A method performed by one or more processors, the method comprising:
receiving information about tissue or cell areas of a single digital pathology image; and
visually representing each of the tissue or cell areas as a proportion of all of the tissue or cell areas using one or more respective nested, interactive areas located entirely within a single area, the nested areas proportional to the respective proportions of the tissue or cell areas.

2. The method of claim 1, wherein for plural tissue areas, further comprising visually representing each of the tissue areas or cell content percentages with a respective one of the nested areas and associating each of the nested areas with a respective color.

3. The method of claim 2, wherein the color used for one of the nested areas is a staining color that is different than the color used for another of the nested areas.

4. The method of claim 2, further comprising visually presenting an annotation associated with each of the colored nested areas, the annotation indicating a tissue type or cell type.

5. The method of claim 4, wherein each of the annotations are part of a legend for the visual representations.

6. The method of claim 1, further comprising presenting information about each of the tissue areas responsive to receiving a user input corresponding to one or more of the nested areas.

7. The method of claim 6, wherein the user input comprises one of a single input or a hovering input.

8. The method of claim 6, wherein presenting the information comprises presenting hierarchical information within a selected nested area.

9. The method of claim 6, wherein presenting the information comprises presenting additional user interface options.

10. The method of claim 1, further comprising visually representing tissue or cell content information within one or more of the nested areas.

11. The method of claim 10, wherein visually representing the content information comprises using symbols, shapes, patterns, text, numerals, colors, or alphanumerics.

12. The method of claim 1, further comprising repeating the visual representation for information about tissue areas received respectively for a plurality of digital pathology images.

13. A system, comprising:
a display device;
a memory comprising instructions; and
one or more processors configured by the instructions to:
receive information about tissue or cell areas of a single digital pathology image; and
visually represent, on the display device, respective tissue or cell areas as a proportion of all detected tissue or cell areas of the single digital pathology image using respective nested, interactive areas located entirely within a single area, the nested areas proportional to the respective proportions of the tissue areas.

14. The system of claim 13, wherein for plural tissue areas, the one or more processors are further configured by the instructions to visually represent each of the tissue areas with a respective one of the nested areas and associate each of the nested areas with a respective color.

15. The system of claim 14, wherein the color used for one of the nested areas is a staining color that is different that the color used for another of the nested areas, wherein the one or more processors are further configured by the instructions to visually present, on the display device, an annotation associated with each of the colored nested areas, the annotation indicating a tissue or cell type.

16. The system of claim 13, wherein the one or more processors are further configured by the instructions to present, on the display device, information about each of the tissue or cell areas responsive to receiving a user input corresponding to one or more of the nested areas, wherein the user input comprises one of a single input or hovering input.

17. The system of claim 16, wherein the one or more processors are further configured by the instructions to present the information by presenting one or a combination of:
hierarchical information within a selected nested area; or
additional user interface options.

18. The system of claim 13, wherein the one or more processors are further configured by the instructions to visually represent, on the display device, tissue or cell content information using symbols, shapes, patterns, text, numerals, colors, or alphanumerics within one or more of the areas.

19. The system of claim 13, wherein the one or more processors are further configured by the instructions to repeat the visual representation for information about tissue or cell areas received respectively for a plurality of digital pathology images.

20. A non-transitory, computer readable storage medium comprising instructions that, when executed by one or more processors, cause the one or more processors to:
represent, for a visualization, respective tissue or cell areas as a proportion of all detected tissue or cell areas of a single digital pathology image using respective nested, interactive areas located entirely within a single area, the nested areas proportional to the respective proportions of the tissue areas.
